(19) United States
(12) Patent Application Publication
(10) Pub. No.: US 2010/0303737 A1
(43) Pub. Date: Dec. 2, 2010

(54) Tooth-Cleaning Formulation

(76) Inventor: Sven HURTIG, Chemnitz (DE)

Correspondence Address: CROWELL & MORING LLP;INTELLECTUAL PROPERTY GROUP, P.O. BOX 14300, WASHINGTON, DC, 20044-4300, US

(21) Appl. No.: 12/792,278

(22) Filed: Jun. 2, 2010

(30) Foreign Application Priority Data
Jun. 2, 2009 (DE) DE202009007704.7

(51) Int. Cl. A61K 8/98; A61K 8/26; A61K 8/25; A61K 8/20; A61K 8/97; A61K 8/24; A61K 8/21; A61K 9/68; A61Q 11/00

(52) U.S. Cl. 424/48; 424/49; 424/58; 424/57; 424/52

(57) ABSTRACT

A tooth-cleaning formulation which contains Himalaya salt in an unadulterated and also non-heat-treated form and/or Sango coral, with the Himalaya salt being used as a direct natural raw material and not being heated and sterilized itself.

### FIELD OF THE INVENTION

The invention relates to a tooth-cleaning formulation which has outstanding cleaning effect and contributes in total to the improvement of oral hygiene.

### BACKGROUND OF THE INVENTION

A toothpaste with alumina is known from EP 327,776 B1 (=DE 38 89 895 T2) which consists of a homogeneous mixture of alumina, glycerin and water and can also contain sea salt. A tooth-cleaning formulation by using calcium salt is also known from U.S. Pat. No. 5,843,406 (=DE 699 35 411 T2).

U.S. Pat. No. 4,826,676 (=EP 0 297 563 B1) discloses an aqueous toothpaste formulation which contains a polishing agent, a source for soluble fluoride ions and zinc-exchanged zeolite as a source of zinc ions, with the same being active in the inhibition of the formation of plaque and tartar. The use of a zinc-exchanged zeolite occurs with a molar ratio of SiO₂/Al₂O₃ of 2.5 to 10 as at least 10% of weight of the polishing agent and the setting of the pH value of the composition to over 9.5.

KR 2008 014 399 A discloses that Himalaya salt has a useful effect in toothpaste only in a highly processed form with long-term annealing/melting (1200° C. up to 1300° C.), cooling, grinding and sterilizing. In this case, the input of resources, energy and manpower is not viable in relation to the useful effect. A traditional form of this industrial method has been known for hundreds of years for the production of “bamboo salt” and any kind of alchemical preparation of mineral products. It is hardly understandable why the highly heated salt needs to be sterilized in addition.

DE 100 65 413 A1 defines that a good dental care formulation can make do only and precisely with sweeteners and polyethylene glycol and preservatives and technical fluorine compounds and colorants and pigments and co-enzyme Q₁₀ or/and ubiquinone (derivatives) or/and vitamin A.

A dental care formulation is disclosed in EP 1 965 765 B1 which contains silicon dioxide nanoparticles especially in a “liposomal dispersion” under further use of “glucan ether derivatives”. Very extensive methods that require a high input of energy, resources and human capital are described as necessary for these special process products.

The use of the aforementioned tooth-cleaning formulations is possible, but not optimal for ecological, economic and technical reasons. Despite the extensive efforts of the prior art, there has remained a need for improved tooth-cleaning formulations.

### SUMMARY OF THE INVENTION

It is the object of the present invention to provide a tooth-cleaning formulation which has a good cleaning effect without damaging the dental enamel, protects from tooth decay and periodontitis, and is able to revitalize the enamel.

The inventive step is especially that the components substantially fulfill the inventive purpose in their natural quality, from natural sources and with the lowest amount of technical processing, and all the requirements placed on a marketable product. Resources such as low-energy processing have highest priority in respect of nature and man.

This and other objects are achieved by the invention as broadly described and claimed hereinafter. Advantageous preferred embodiments are also described and claimed.

In accordance with the invention, the tooth-cleaning formulation contains unadulterated Himalaya salt which is not heat-treated, and/or Sango coral. The Himalaya salt is used as a directly gained natural raw material and does not require any further industrial treatment. It is not heated, chemically purified and sterilized.

Himalaya salt is also known as Himalaya crystal salt, Himalaya rock salt, crystal salt from the base of the Himalaya, salt range rock salt, crystal salt, crystal salt from the salt range of the Himalaya, crystal salt from the salt mountains, Indus salt, Himalaya Indus salt, Indus salt from the Himalaya, Kaiser salt, Alexander salt, Tibet salt, Hunza salt, Hunza crystal salt, christian-all salt, ayurvedic magic salt, Himalayan rock salt, salt range rock salt, halit crystal salt, Himalayan halit salt, crystal salt pin, Himalayan pink salt, Himalayan crystal salt Indus Himalaya salt.

The Sango corals are subdivided into Sango sea corals and Sango land corals, which are both suitable for the application in accordance with the invention.

Zeolite and/or quartz and/or an organic silicon source and/or an organic calcium source and/or potassium chloride and plant additives (herbs) such as sage, rosemary, tea-tree, clove, fennel, cinnamon, mint and types of mint, eucalyptus, chamomile, horsetail, neem, Echinacea, anise, myrrh, thyme, lemon can be added to the tooth-cleaning formulation in accordance with the invention individually or in combination, as well as foaming agents (tensides).

The components of Himalaya salt, Sango coral and zeolite, quartz, organic silicon source, organic calcium source and potassium chloride can be ground in a fine manner, especially micro-fine manner. Furthermore, the Himalaya salt can be used in the form of brine. For this purpose, the unadulterated Himalaya salt is dissolved in water. The water or finished brine can be heated to 120° C., if required.

Furthermore, the tooth-cleaning formulation may contain silicic acid and chalk and/or bicarbonate (sodium hydrogen carbonate, sodium bicarbonate). In addition, glycerin and/or sorbitol and/or xylitol and/or mannitol may be used in the tooth-cleaning formulation.

As is common practice in dental care, the tooth-cleaning formulation in accordance with the invention can contain one or several fluoride sources such as sodium fluoride, sodium monofluorophosphate or potassium fluoride. Preferably, natural fluoride sources such as fluorspar (calcium fluoride/CaF₂) are used.

Furthermore, the tooth-cleaning formulation in accordance with the invention may contain one or more sources of phosphate, preferably alkali metal phosphates such as sodium polyphosphate.

The tooth-cleaning formulation in accordance with the invention can be present in the form of tooth salt, tooth powder, tooth paste, tooth gel or tooth-cleaning chewing gum or mouthwash.

The tooth-cleaning formulation in the form of tooth salt or tooth powder consists of Himalaya salt and/or Sango coral which is preferably laced with herbs and/or further cleaning grains such as siliceous earth/silica, kaolin, sodium polyphosphate, zeolite, quartz, chalk, bicarbonate and also mineral clay.

The toothpaste or tooth gel is especially an aqueous mixture or emulsion of

| | |
|---|---|
| 0.9-60% | water and/or Himalaya brine |
| 1.0-40% | silicic acid |
| 0.1-50% | chalk and/or bicarbonate |
| 1.0-60% | glycerin and/or sorbitol and/or xylitol and/or mannitol |
| 0.1-5% | xanthan gum and/or alginate |
| 0.1-40% | Himalaya salt and/or Sango coral, |

to which can also be added various herbs and foaming agents (tensides) such as sodium cocoyl glutamate and disodium cocoyl glutamate (coconut tensides) or lauryl glucoside (sugar tenside).

The basic mixture can also contain zeolite (e.g. 0.1 to 40%) and/or quartz (e.g. 0.1 to 40%) and/or an organic silicon source (e.g. 0.1 to 40%) and/or an organic calcium source (e.g. 0.1 to 40%) and/or potassium chloride (e.g. 0.1 to 12%).

The components listed here are combined into a toothpaste or tooth gel, in that the powdery components, including the unadulterated Himalaya salt and/or Sango corals, are mixed slowly. Thereafter the desired composition of the moisture carriers is introduced, thus allowing slurrying of the existing formulation. The further secure processing capability (freedom of dust, transport capability) is thus ensured. Water and/or Himalaya brine is added in a further process step, and also the mixture of herbs and flavor carriers. This is followed by a final homogenization.

Another procedure is also possible: All fluid components are mixed together first and the powdery components are then added, including the unadulterated Himalaya salt and/or Sango corals. The flavor carriers are added finally. In both variants, the mixing process can occur under vacuum, if desired.

When the tooth-cleaning formulation is produced in the form of a mouthwash, then this is preferably 0.9 to 27% brine solution made of Himalaya salt, optionally with additives in the form of extracts from different herbs.

The pH-value of the tooth-cleaning formulation can preferably be 7 to 8.5.

The toothpaste in accordance with the invention cleans the teeth and strengthens the gums especially through a combination of Himalaya salt and different medicinal plants. Mineral cleaning grains made of chalk, bicarbonate and salt crystals remove plaque in an exceptionally careful way.

Himalaya salt is exceptionally rich in minerals such as calcium, magnesium, sodium, potassium, sulfate and contains further trace elements. Fluoride, which is important for the teeth, is also contained in a naturally bound form. It acts antibacterial, stimulates blood circulation, promotes natural salivation, supports self-cleaning of the teeth and neutralizes damaging acids.

The calcium of the Sango corals mineralizes the enamel. The Sango coral, which is also known as Sango calcium, provides a natural complex of minerals because it contains all essential minerals and further counteracts hyperacidity.

Zeolites from the zeolite group from a diverse family of chemically complex silicate minerals. It has been found that microfine zeolite is especially suitable as a cleaning body, binds contaminants and heavy metals and can neutralize bad breath.

Extracts from different herbs in the form of extracts, tinctures or natural essential oils strengthen the gums and oral flora, and exert an anti-inflammatory, antiseptic and/or antibacterial way activity.

The invention will be explained in further detail hereinafter with reference to illustrative embodiments set forth in the following examples.

### EXAMPLE 1

In order to produce the toothpaste, an aqueous solution (paste) is made of 0.9 to 60% of water, 1 to 40% of silicic acid, 0.1 to 50% chalk and/or bicarbonate, 1 to 60% of glycerin and/or sorbitol and/or xylitol and/or mannitol, and 0.1 to 5% of xanthan gum and/or alginate as well as 0.1 to 40% of Himalaya salt and/or Sango coral. Sage, rosemary and tea-tree as well as horsetail, neem, thyme and myrrh are added to this basic substance. A toothpaste is created which forms natural protection against tooth decay and periodontitis and prevents bleeding gums. The toothpaste acts in an anti-inflammatory, antiseptic and antibacterial manner and can be used in dentistry for the healing of wounds. The tea-tree oil prevents inflammation and is especially suitable in the case of sensitive gums. A source of fluoride can be used in addition if necessary.

### EXAMPLE 2

Extracts of the herbs clove, fennel and cinnamon, supplemented by horsetail, neem, myrrh and Echinacea, are added to the basic composition of Example 1, through which the toothpaste also offers natural protection from tooth decay and periodontitis. This toothpaste also acts in an antibacterial, antiseptic and anti-inflammatory way. The application of cloves supports pain relief and Echinacea the defensive function of the oral flora. The menthol-free toothpaste is suitable for homeopathic treatment. A source of fluoride can be used in addition if required.

### EXAMPLE 3

An extract of herbs of mint and types of mint, eucalyptus and chamomile, supplemented by horsetail, neem, sage and myrrh, is added to the basic mixture consisting of 0.9 to 60% of water and/or Himalaya brine, 1 to 40% of silicic acid, 0.1 to 50% chalk and/or bicarbonate, 1 to 60% of glycerine and/or sorbitol and/or xylitol and/or mannitol, and 0.1 to 5% of xanthan gum and/or alginate as well as 0.1 to 40% of Himalaya salt and/or Sango coral. A source of phosphate and/or fluoride can be used in addition if necessary. The toothpaste also acts in an antibacterial, antiseptic and anti-inflammatory way. Chamomile prevents bleeding of the gums and is used in dentistry for the healing of wounds. Natural protection from tooth decay and periodontitis is improved. Mint and menthol ensure pleasant taste and fresh breath.

### EXAMPLE 4

Only Sango coral (0.1 to 40%), zeolites (0.1 to 40%), quartz (0.1 to 40%) are added to the basic mixture of example 3 instead of the powdery Himalaya salt, and if required potassium chloride (0.1 to 12%), a source of organic silicon (0.1 to 40%), a further source of organic calcium (0.1 to 40%), a source of phosphate (0.1 to 12%), a source of fluoride (0.1 to 12%) and only Himalaya brine (0.9 to 60%) instead of water.

This novel tooth gel mineralizes the teeth and strengthens the gums by a combination of Himalaya brine and natural minerals for example. Cleaning bodies made of chalk, silicic acid and zeolite remove plaque in a careful way. Siliceous earth, which is also known as terra silicea, strengthens the connective tissue and the teeth. Zeolite ground in a microfine manner binds contaminants and heavy metals and neutralizes bad breath. The active agent of potassium chloride enters the tooth through dentine canals and forms a protective jacket around the dental nerve. The tooth is calmed and sensitivity to pain is reduced. The calcium of the Sango coral, and optionally further sources of organic calcium, and a source of fluoride, preferably natural calcium fluoride (fluorspar) remineralize the enamel. Additional extracts added to the tooth gel such as those of horsetail, neem, Echinacea, sage, myrrh, anise, curled mint (menthol-free) and lemon strengthen the gums and oral flora, and act in an antibacterial, antiseptic and anti-inflammatory way. The quartz powder that is ground in a microfine manner and optionally a source of organic silicon vitalize and strengthen teeth, gums and oral flora. This tooth gel also offers natural protection from tooth decay and periodontitis in the case of regular application. The tooth gel in Example 4 is free from menthol and is suitable for homeopathic treatment.

### EXAMPLE 5

A further embodiment of the tooth-cleaning formulation is represented by the following tooth-cleaning chewing gum. The chewable mass preferably consists of:

| | |
|---|---|
| 10-40% | gum base |
| 10-70% | xylitol and/or mannitol |
| 1-50% | glycerin and/or sorbitol |
| 0.1-12% | Himalaya salt and/or Sango coral |
| 0.1-5% | herbal extracts and/or spice extracts |
| 0-3.2% | organic thickeners |

A source of phosphate such as sodium polyphosphate and further mineral carriers and cleaning bodies such as siliceous earth/silica, kaolin, quartz, chalk, bicarbonate, and a source of organic silicon and also a source of organic calcium can optionally be added to the basic mixture.

It can be determined in summary that with the present invention it has been managed to produce a tooth-cleaning formulation with Sango coral and/or Himalaya salt in a manner that saves resources and energy, without having to heat the Himalaya salt to very high temperatures, to make it melt or to sterilize it. Instead, it can be used directly as an unadulterated raw material. The ecological and economic factors were improved with the tooth-cleaning formulation in accordance with the invention with regard to effectiveness.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A tooth-cleaning formulation comprising Himalaya salt in an unadulterated and also non-heat-treated form and/or Sango coral, with the Himalaya salt being used as a direct natural raw material and not being heated and sterilized itself.

2. A tooth-cleaning formulation according to claim 1, wherein the Himalaya salt and/or Sango coral is present in the form of a powder ground in a fine and or microfine manner.

3. A tooth-cleaning formulation according to claim 1, wherein the Himalaya salt and/or Sango coral is present in the form of a brine.

4. A tooth-cleaning formulation according to claim 3, wherein the brine is formed by dissolving the unadulterated Himalaya salt in water, and optionally heating the water or finished brine to a temperature of 120° C.

5. A tooth-cleaning formulation according to claim 1, further comprising at least one powdered substance selected from the group consisting of zeolites, quartz, organic silicon sources, organic calcium sources and potassium chloride.

6. A tooth-cleaning formulation according to claim 5, wherein said at least one powdered substance is ground in a microfine manner.

7. A tooth-cleaning formulation according to claim 1, further comprising at least one herb selected from the group consisting of sage, rosemary, tea-tree, clove, fennel, cinnamon, mint, eucalyptus, chamomile, horsetail, neem, Echinacea, anise, myrrh, thyme, and lemon.

8. A tooth-cleaning formulation according to claim 1, further comprising at least one additive selected from the group consisting of silicic acid, chalk, sodium bicarbonate, glycerin, sorbitol, xylitol, and mannitol.

9. A tooth-cleaning formulation according to claim 1, further comprising at least one mineral source selected from the group consisting of phosphate sources and fluoride sources.

10. A tooth-cleaning formulation according to claim 9, wherein said phosphate sources are selected from the group consisting of sodium polyphosphate and dicalcium phosphate, and said fluoride sources are selected from the group consisting of fluorspar, calcium fluoride, sodium fluoride, sodium monofluorophosphate, and potassium fluoride.

11. A tooth-cleaning formulation according to claim 1, wherein said formulation is in the form of a tooth salt, tooth powder, dental cream, toothpaste, tooth gel, tooth-cleaning chewing gum or mouthwash.

12. A tooth-cleaning formulation according to claim 1, wherein said formulation is a tooth salt or tooth powder made of Himalaya salt and/or Sango coral and further comprises further cleaning bodies selected from the group silica, siliceous earth, kaolin, sodium polyphosphate, zeolites, quartz, chalk, bicarbonate and mineral clay.

13. A tooth-cleaning formulation according to claim 12, further comprising at least one herb.

14. A tooth-cleaning formulation according to claim 1, wherein said formulation is a toothpaste or tooth gel comprising a mixture of:

| | |
|---|---|
| 0.9-60% | water and/or Himalaya salt brine; |
| 1.0-40% | silicic acid; |
| 0.1-50% | chalk and/or bicarbonate |
| 1.0-60% | glycerin and/or sorbitol and/or xylitol and/or mannitol; |
| 0.1-5% | xanthan gum and/or alginate; |
| 0.1-40% | Himalaya salt and/or Sango coral. |

15. A tooth-cleaning formulation according to claim 14, further comprising a tenside.

16. A tooth-cleaning formulation according to claim 15, wherein said tenside is selected from the group consisting of sodium cocoyl glutamate, disodium cocoyl glutamate, and lauryl glucosides.

17. A tooth-cleaning formulation according to claim 14, further comprising at least one substance selected from the group consisting of:
0.1 to 40% of zeolite
0.1 to 40% of quartz
0.1 to 40% of a source of organic silicon
0.1 to 40% of a source of organic calcium, and
0.1 to 12% of potassium chloride.

18. A tooth-cleaning formulation according to claim 1, wherein said formulation is a tooth-cleaning chewing gum comprising:

| | |
|---|---|
| 10-40% | gum base |
| 10-70% | xylitol and/or mannitol |
| 1-50% | glycerin and/or sorbitol |
| 0.1-12% | Himalaya salt and/or Sango coral |
| 0.1-5% | herbal extracts and/or spice extracts |
| 0-3.2% | organic thickeners. |

19. A tooth-cleaning formulation according to claim 18, further comprising a source of phosphate or at least one mineral carrier or cleaning body selected from the group consisting of silica, siliceous earth, kaolin, quartz, chalk, sodium bicarbonate, sources of organic silicon, and sources of organic calcium.

20. A tooth-cleaning formulation according to claim 1, wherein said formulation is a mouthwash comprising a 0.9 to 27% brine solution made of Himalaya salt, and optionally at least one herb.
